(19) United States
(12) Patent Application Publication
(10) Pub. No.: US 2025/0236718 A1
(43) Pub. Date: Jul. 24, 2025

(54) METHOD FOR MANUFACTURING BISPHENOL A

(71) Applicant: National Taiwan University, Taipei City (TW)

(72) Inventors: Chia-Wen WU, Taipei City (TW); Wei-Sheng LIAO, Taipei City (TW); Philip Anggo KRISBIANTORO, Taipei City (TW); Tzu-Ming LIN, Taipei City (TW)

(21) Appl. No.: 18/808,967

(22) Filed: Aug. 19, 2024

Related U.S. Application Data
(60) Provisional application No. 63/624,402, filed on Jan. 24, 2024.

(51) Int. Cl. C08J 11/24; C07C 37/82

(52) U.S. Cl. CPC C08J 11/24; C07C 37/82; C08J 2369/00

(57) ABSTRACT

A method for manufacturing a bisphenol A (BPA) includes subjecting a polycarbonate and an aliphatic monohydric alcohol to a depolymerization reaction in the presence of sodium aluminate and a solvent, so as to obtain a first crude product containing the BPA. The BPA is purified from the first crude product by subjecting the first crude product to a solid-liquid separation treatment and a removal treatment in sequence.

### CROSS-REFERENCE TO RELATED APPLICATION

This application claims the benefit of U.S. Provisional Patent Application No. 63/624,402, filed on Jan. 24, 2024, the entire disclosure of which is incorporated by reference herein.

### FIELD

The present disclosure relates to a method for recycling and processing a polymer, and more particularly to a method for manufacturing a bisphenol A (BPA) by depolymerizing a polycarbonate.

### BACKGROUND

In recent years, many countries have placed great emphasis on the recycling and reuse of polycarbonate wastes. At present, the mainstream method for recycling the polycarbonate wastes is a chemical recycling method, which has low purity requirements for the polycarbonate wastes, and the bisphenol A (BPA) obtained by the chemical recycling method can be reused.

The chemical recycling method, such as a pyrolysis method, a hydrolysis method, an alcoholysis method, an ammonolysis method, or a hydrogenolysis method, is utilized to depolymerize a polycarbonate in the polycarbonate wastes so as to obtain the BPA. Among the aforesaid chemical recycling methods, the alcoholysis method is the most promising due to its higher yield of the BPA.

In the alcoholysis method, the polycarbonate is subjected to a depolymerization reaction with an alcohol in the presence of a catalyst for a catalytic reaction, so as to obtain the BPA. The catalytic reaction includes a homogeneous catalysis reaction and a heterogeneous catalysis reaction. The homogeneous catalysis reaction is a type of a catalytic reaction in which a catalyst is in a phase same as that of the reactants, typically in a liquid or gas phase. Therefore, in the homogeneous catalysis reaction, the catalyst and the reactants are uniformly mixed, which often leads to a catalytic reaction that is relatively efficient but difficult separation of the catalyst from a resultant product. The catalyst in the homogeneous catalysis reaction may be an alkali metal alkoxide (e.g., sodium methoxide).

The heterogeneous catalysis reaction is a type of a catalytic reaction in which a catalyst is in a phase different from that of the reactants. Typically, the catalyst is a solid, while the reactants are gases or liquids. Therefore, in the heterogeneous catalysis reaction, the catalyst has an advantage of being easily separated and being reused. CN 114904542 A discloses a composite catalyst and a method for depolymerizing a polycarbonate plastic by a heterogeneous catalysis reaction using the composite catalyst. The composite catalyst is a supported catalyst, and includes a basic metal oxide serving as a carrier, a transition metal salt serving as an active component, and an alkali metal alkoxide serving as an auxiliary agent. The basic metal oxide is selected from the group consisting of magnesium oxide (MgO), calcium oxide (CaO), copper (II) oxide (CuO), manganese dioxide (MnO₂), iron (III) oxide (Fe₂O₃), iron (II) oxide (FeO), cobalt (II) oxide (CoO), cobalt (II,III) oxide (Co₃O₄), and combinations thereof mixed in any proportion. The transition metal salt is selected from the group consisting of iron chloride, cobalt chloride, nickel chloride, copper chloride, zinc chloride, manganese chloride, a nitrate salt, an acetate salt, a sulfate salt, an acetylacetonate salt, and combinations thereof mixed in any proportion. The alkali metal alkoxide is selected from the group consisting of potassium methoxide, sodium methoxide, potassium ethoxide, sodium ethoxide, potassium isopropoxide, sodium n-propoxide, sodium isopropoxide, potassium t-butoxide, sodium n-butoxide, sodium t-butoxide, potassium t-pentoxide, sodium t-pentoxide, and combinations thereof mixed in any proportion. The method of CN 114904542 A includes dissolving the polycarbonate plastic in an organic solvent or without dissolving the polycarbonate plastic in any organic solvent, followed by subjecting the polycarbonate plastic and an alcohol to a depolymerization reaction at a temperature ranging from 80° C. to 100° C. for a time period ranging from 3 hours to 10 hours in the presence of the composite catalyst, so as to obtain a BPA.

In view of the aforesaid, there is still a need to develop an effective way for manufacturing a bisphenol A (BPA).

### SUMMARY

Therefore, an object of the present disclosure is to provide a method for manufacturing a bisphenol A (BPA), which can alleviate at least one of the drawbacks of the prior art.

According to the present disclosure, the method includes subjecting a polycarbonate and an aliphatic monohydric alcohol to a depolymerization reaction in the presence of sodium aluminate and a solvent, so as to obtain a first crude product containing the BPA.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the X-ray diffraction (XRD) patterns of the bisphenol A (BPA) of Example 1 (EX1) and the commercially available BPA.

FIG. 2 shows the Fourier-transform infrared (FTIR) spectra of the BPA of EX1 and the commercially available BPA.

FIG. 3 shows the differential scanning calorimetry (DSC) curve and the thermogravimetric analysis (TGA) curve of the BPA of EX1.

FIG. 4 shows the proton nuclear magnetic resonance (¹H-NMR) spectrum of the BPA of EX1.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for manufacturing a bisphenol A (BPA), which includes subjecting a polycarbonate and an aliphatic monohydric alcohol to a depolymerization reaction in the presence of sodium aluminate and a solvent, so as to obtain a first crude product containing the BPA.

According to the present disclosure, the first crude product contains, in addition to the BPA, the sodium aluminate, the solvent, and an unreacted aliphatic monohydric alcohol. In certain embodiments, the BPA is purified from the first crude product by the steps of: subjecting the first crude product to a solid-liquid separation treatment, so as to obtain the sodium aluminate and a second crude product containing the BPA, the solvent, and the unreacted aliphatic monohydric alcohol; and subjecting the second crude product to a removal treatment to remove the solvent and the unreacted aliphatic monohydric alcohol from the second crude product, so as to purify the BPA.

In certain embodiments, an example of the polycarbonate may include, but is not limited to, a polycarbonate waste.

According to the present disclosure, the sodium aluminate is a catalyst commonly used in a heterogeneous catalysis reaction, so that the solid-liquid separation treatment can be used to separate the sodium aluminate from the first crude product, and the thus obtained sodium aluminate can be recycled for reuse. In certain embodiments, the sodium aluminate is present in an amount ranging from 0.01 parts by weight to 0.20 parts by weight, based on a total amount of the polycarbonate as 1.0 part by weight. In some exemplary embodiments, the sodium aluminate is present in an amount ranging from 0.01 parts by weight to 0.06 parts by weight, based on a total amount of the polycarbonate as 1.0 part by weight.

According to the present disclosure, the aliphatic monohydric alcohol is selected from the group consisting of methanol, ethanol, and a combination thereof. In certain embodiments, the aliphatic monohydric alcohol is methanol. In certain embodiments, the aliphatic monohydric alcohol is present in an amount ranging from 0.3 parts by weight to 2.4 parts by weight, based on a total amount of the polycarbonate as 1.0 part by weight. In some exemplary embodiments, the aliphatic monohydric alcohol is present in an amount ranging from 0.3 parts by weight to 2.0 parts by weight, based on a total amount of the polycarbonate as 1.0 part by weight.

According to the present disclosure, the solvent may dissolve the polycarbonate, enhance mass transfer, and deliver the aliphatic monohydric alcohol and the sodium aluminate to a molecular chain of the polycarbonate, thereby allowing the depolymerization reaction to proceed. The solvent may be selected based on the Hansen solubility parameter to match a polarity of the polycarbonate.

According to the present disclosure, the solvent is selected from the group consisting of tetrahydrofuran (THF), chloroform, dimethyl carbonate (DMC), dichloromethane (DCM), acetone, acetonitrile, heptane, cyclohexane, and combinations thereof. In certain embodiments, the solvent is THF. In certain embodiments, the solvent is chloroform. In certain embodiments, the solvent is DMC. In certain embodiments, the solvent is DCM. In certain embodiments, the solvent is acetone. In certain embodiments, the solvent is acetonitrile. In certain embodiments, the solvent is heptane. In certain embodiments, the solvent is cyclohexane. In certain embodiments, the solvent is present in an amount ranging from 0.6 parts by weight to 2.7 parts by weight, based on a total amount of the polycarbonate as 1.0 part by weight. In some exemplary embodiments, the solvent is present in an amount ranging from 1.0 part by weight to 2.7 parts by weight, based on a total amount of the polycarbonate as 1.0 part by weight.

In some exemplary embodiments, the sodium aluminate is present in an amount ranging from 0.01 parts by weight to 0.06 parts by weight, the aliphatic monohydric alcohol is present in an amount ranging from 0.3 parts by weight to 2.0 parts by weight, and the solvent is present in an amount ranging from 1.0 part by weight to 2.7 parts by weight, based on a total amount of the polycarbonate as 1.0 part by weight.

According to the present disclosure, the depolymerization reaction is conducted at a temperature ranging from 25.0° C. to 64.7° C. In certain embodiments, the depolymerization reaction is conducted at a temperature ranging from 25.0° C. to 60.0° C. When the temperature ranges from 25.0° C. to 64.7° C. and the aliphatic monohydric alcohol is methanol and/or ethanol, the depolymerization reaction can be conducted at atmospheric pressure (i.e., 1 atm) without utilizing a high-pressure reactor since a range of such temperature is below boiling points of methanol and ethanol, thereby reducing complexity of procedures for the depolymerization reaction and increasing safety.

In certain embodiments, the depolymerization reaction is conducted at a pressure of 1 atm.

In certain embodiments, the depolymerization reaction is conducted for a time period ranging from 0.25 hours to 9.00 hours.

In some exemplary embodiments, the depolymerization reaction is conducted at a temperature ranging from 25.0° C. to 60.0° C. and a pressure of 1 atm for a time period ranging from 0.25 hours to 9.00 hours.

In certain embodiments, an example of the solid-liquid separation treatment may include, but is not limited to, a suction filtration treatment.

In certain embodiments, an example of the removal treatment may include, but is not limited to, a rotary evaporation treatment.

The disclosure will be further described by way of the following examples. However, it should be understood that the following examples are solely intended for the purpose of illustration and should not be construed as limiting the disclosure in practice.

### EXAMPLES

### Example 1 (EX1)

The materials, the amounts thereof, and the operating conditions for conducting the depolymerization reaction in the method for manufacturing the bisphenol A (BPA) of EX1 are shown in Tables 1 and 2 below. First, 5 parts by weight of polycarbonate (Manufacturer: Wanhua Chemical Group Co., Ltd., Model no.: A1107) and 5 parts by weight of methanol were subjected to a depolymerization reaction by stirring at 25° C. for 9 hours under atmospheric pressure (i.e., 1 atm) in the presence of 10.0 parts by weight of tetrahydrofuran (THF, serving as a solvent) and 0.05 parts by weight of sodium aluminate (serving as a catalyst for a heterogeneous catalysis reaction), so as to obtain a first crude product containing a bisphenol A (BPA), the sodium aluminate, the THF, and an unreacted methanol. Next, the first crude product was subjected to a suction filtration treatment (serving as a solid-liquid separation treatment), so as to obtain the sodium aluminate and a second crude product containing the BPA, the THF, and the unreacted methanol. Thereafter, the second crude product was subjected to a rotary evaporation treatment (serving as a removal treatment) using a rotary concentrator to remove the THF and the unreacted methanol from the second crude product, so as to purify the BPA of EX1.

### Examples 2 to 35 (EX2 to EX35)

The procedures and conditions in the method for manufacturing the BPA of a respective one of EX2 to EX35 were similar to those of EX1, except that the amounts of the sodium aluminate and methanol, the amount and type of the solvent, and the operating conditions for conducting the depolymerization reaction were varied as shown in Tables 1 and 2 below.

TABLE 1

| | Amount of | Amount of | Amount of | Amount of |
|---|---|---|---|---|
| | polycarbonate | sodium | methanol | solvent |
| | (parts by | aluminate | (parts by | (parts by |
| | weight) | (parts by weight) | weight) | weight) |
| EX1 | 5 | 0.05 | 5 | 10 |
| EX2 | 5 | 0.1 | 5 | 10 |
| EX3 | 5 | 0.2 | 5 | 10 |
| EX4 | 5 | 0.3 | 5 | 10 |
| EX5 | 5 | 0.2 | 5 | 10 |
| EX6 | 5 | 0.2 | 5 | 10 |
| EX7 | 5 | 0.2 | 5 | 10 |
| EX8 | 5 | 0.2 | 5 | 10 |
| EX9 | 5 | 0.2 | 5 | 10 |
| EX10 | 5 | 0.2 | 5 | 10 |
| EX11 | 5 | 0.2 | 5 | 10 |
| EX12 | 5 | 0.2 | 5 | 10 |
| EX13 | 5 | 0.2 | 5 | 10 |
| EX14 | 5 | 0.2 | 5 | 10 |
| EX15 | 5 | 0.2 | 5 | 10 |
| EX16 | 5 | 0.2 | 5 | 10 |
| EX17 | 5 | 0.2 | 5 | 10 |
| EX18 | 5 | 0.2 | 5 | 10 |
| EX19 | 5 | 0.2 | 5 | 10 |
| EX20 | 5 | 0.2 | 5 | 10 |
| EX21 | 5 | 0.2 | 5 | 10 |
| EX22 | 5 | 0.2 | 5 | 10 |
| EX23 | 5 | 0.2 | 5 | 10 |
| EX24 | 5 | 0.2 | 5 | 10 |
| EX25 | 5 | 0.2 | 10 | 5 |
| EX26 | 5 | 0.2 | 7.5 | 7.5 |
| EX27 | 5 | 0.2 | 2.5 | 12.5 |
| EX28 | 5 | 0.2 | 1.5 | 13.5 |
| EX29 | 5 | 0.2 | 5 | 10 |
| EX30 | 5 | 0.2 | 5 | 10 |
| EX31 | 5 | 0.2 | 5 | 10 |
| EX32 | 5 | 0.2 | 5 | 10 |
| EX33 | 5 | 0.2 | 5 | 10 |
| EX34 | 5 | 0.2 | 5 | 10 |
| EX35 | 5 | 0.2 | 5 | 10 |

TABLE 2

| | | Operating | Time period for | Operating |
|---|---|---|---|---|
| | Type of | temperature | depolymerization | pressure |
| | solvent | (° C.) | reaction (hour) | (atm) |
| EX1 | THF | 25 | 9 | 1 |
| EX2 | THF | 25 | 9 | 1 |
| EX3 | THF | 25 | 9 | 1 |
| EX4 | THF | 25 | 9 | 1 |
| EX5 | THF | 25 | 0.25 | 1 |
| EX6 | THF | 25 | 0.5 | 1 |
| EX7 | THF | 25 | 1 | 1 |
| EX8 | THF | 25 | 3 | 1 |
| EX9 | THF | 25 | 6 | 1 |
| EX10 | THF | 40 | 0.25 | 1 |
| EX11 | THF | 40 | 0.5 | 1 |
| EX12 | THF | 40 | 1 | 1 |
| EX13 | THF | 40 | 2 | 1 |
| EX14 | THF | 40 | 3 | 1 |
| EX15 | THF | 40 | 5 | 1 |
| EX16 | THF | 40 | 7 | 1 |
| EX17 | THF | 50 | 0.5 | 1 |
| EX18 | THF | 50 | 1 | 1 |
| EX19 | THF | 50 | 2 | 1 |
| EX20 | THF | 50 | 3 | 1 |
| EX21 | THF | 60 | 0.25 | 1 |
| EX22 | THF | 60 | 0.5 | 1 |
| EX23 | THF | 60 | 1 | 1 |
| EX24 | THF | 60 | 2 | 1 |
| EX25 | THF | 25 | 9 | 1 |
| EX26 | THF | 25 | 9 | 1 |
| EX27 | THF | 25 | 9 | 1 |
| EX28 | THF | 25 | 9 | 1 |
| EX29 | Heptane | 25 | 9 | 1 |
| EX30 | Cyclohexane | 25 | 9 | 1 |
| EX31 | Chloroform | 25 | 9 | 1 |
| EX32 | DMC | 25 | 9 | 1 |
| EX33 | DCM | 25 | 9 | 1 |
| EX34 | Acetone | 25 | 9 | 1 |
| EX35 | Acetonitrile | 25 | 9 | 1 |

### Property Evaluation

### A. Calculation of Bisphenol a (BPA) Yield

The BPA yield in the method of a respective one of EX1 to EX35 was calculated using the following Equation (1):

$$A = [ ( {W}_{BPA} / {MW}_{BPA} ) / ( {W}_{PC} / {MW}_{PC} ) ] \times 100 \% \qquad ( 1 )$$

where
A=BPA yield (%)
W_{BPA}=weight of BPA in each example
MW_{BPA}=molecular weight of BPA (i.e., 228.29 g/mol)
W_{PC}=weight of polycarbonate in each example
MW_{PC}=molecular weight of repeat unit of polycarbonate (i.e., 254.3 g/mol)

The results are shown in Table 3 below.

TABLE 3

| | EX1 | EX2 | EX3 | EX4 | EX5 | EX6 | EX7 |
|---|---|---|---|---|---|---|---|
| BPA yield (%) | 38.0 | 55.5 | 92.1 | 97.6 | 16.4 | 18.7 | 26.5 |
| | EX8 | EX9 | EX10 | EX11 | EX12 | EX13 | EX14 |
| BPA yield (%) | 41.8 | 56.8 | 27.2 | 33.9 | 44.8 | 53.3 | 75.3 |
| | EX15 | EX16 | EX17 | EX18 | EX19 | EX20 | EX21 |
| BPA yield (%) | 88.9 | 96.9 | 51.9 | 63.4 | 76.4 | 94.0 | 46.6 |
| | EX22 | EX23 | EX24 | EX25 | EX26 | EX27 | EX28 |
| BPA yield (%) | 70.4 | 88.6 | 96.8 | 10.6 | 34.0 | 96.0 | 91.9 |
| | EX29 | EX30 | EX31 | EX32 | EX33 | EX34 | EX35 |
| BPA yield (%) | 0.6 | 0.6 | 23.5 | 39.6 | 41.4 | 31.0 | 29.5 |

Referring to Tables1 to 3, by virtue of using the sodium aluminate as a catalyst for a heterogeneous catalysis reaction to catalyze the depolymerization reaction of the polycarbonate with the aliphatic monohydric alcohol (i.e., methanol) at the temperature ranging from 25° C. to 60° C. for the time period ranging from 0.25 hours to 9 hours under atmospheric pressure (i.e., 1 atm), the BPA can be indeed obtained. To be specific, the method of EX4 can achieve a BPA yield of up to 97.6%. These results indicate that, by virtue of the method for manufacturing the BPA of the present disclosure, the BPA can be obtained using an inexpensive, easily separable and recyclable catalyst (i.e., the sodium aluminate) for the heterogeneous catalysis reaction conducted at room temperature under atmospheric pressure without utilizing a high-pressure reactor, thereby reducing complexity of procedures for the depolymerization reaction and increasing safety.

### B. X-Ray Diffraction (XRD) Analysis

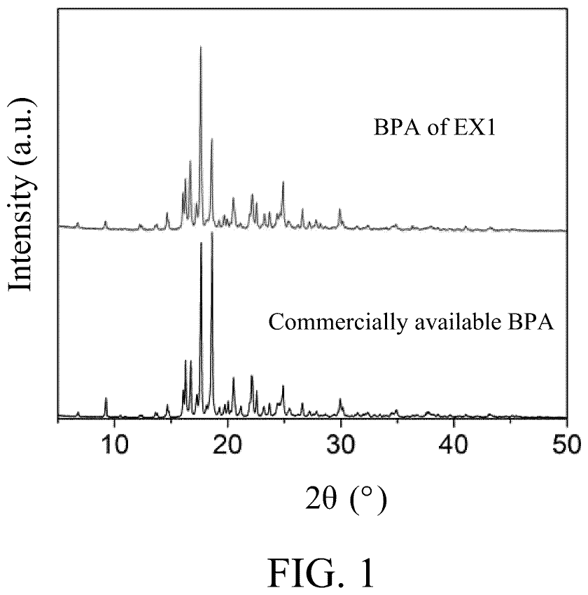

The BPA of EX1 and a commercially available BPA (Manufacturer: Thermo Fisher Scientific Inc., purity: 97%) were subjected to XRD analysis using an X-ray diffractometer (Manufacturer: Rigaku Corporation, Model no.: SmartLab SE), so as to obtain XRD patterns as shown in FIG. 1.

Referring to FIG. 1, the XRD pattern of the BPA of EX1 was substantially consistent with that of the commercially available BPA, demonstrating that the BPA of EX1 had a crystalline structure same as that of the commercially available BPA.

### C. Fourier-Transform Infrared Spectroscopy (FTIR) Analysis

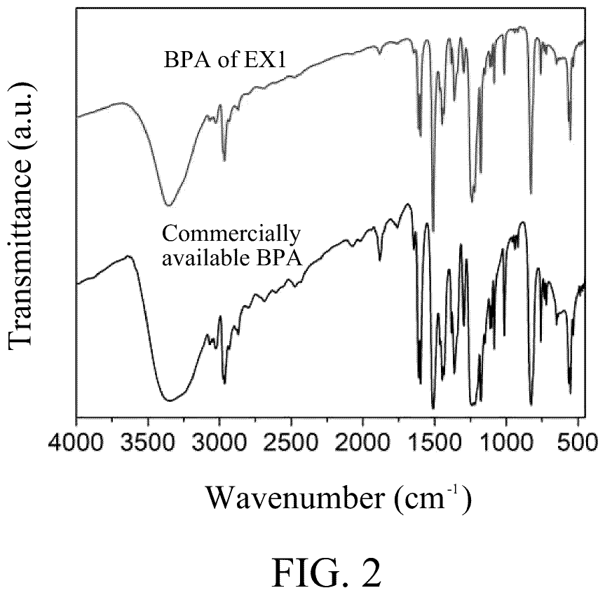

The BPA of EX1 and the commercially available BPA were subjected to FTIR analysis using a FTIR spectrometer (Manufacturer: PerkinElmer Inc., Model no.: Spectrum 100), so as to obtain FTIR spectra as shown in FIG. 2.

Referring to FIG. 2, the FTIR spectrum of BPA of EX1 was substantially consistent with that of the commercially available BPA, demonstrating that the BPA of EX1 had a functional group same as that of the commercially available BPA.

### D. Differential Scanning Calorimetry (DSC) Analysis

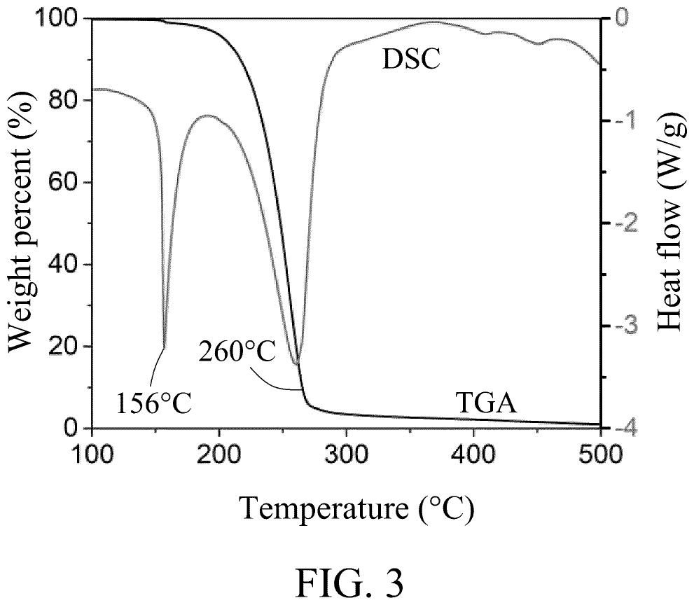

The BPA of EX1 was subjected to DSC analysis using a differential scanning calorimeter (Manufacturer: TA Instruments, Model no.: SDT 650), so as to obtain a DSC curve as shown in FIG. 3.

Referring to FIG. 3, the DSC curve showed that the BPA of EX1 had a melting point of 156° C., which is within the range of the melting point of BPA well known to those skilled in the art.

### E. Thermogravimetric Analysis (TGA)

The BPA of EX1 was subjected to TGA using a thermogravimetric analyzer (Manufacturer: TA Instruments, Model no.: SDT 650), so as to obtain a TGA curve as shown in FIG. 3.

Referring to FIG. 3, the TGA curve showed that the BPA of EX1 had a decomposition temperature of 260° C., which is within the range of the decomposition temperature of BPA well known to those skilled in the art.

### F. Proton Nuclear Magnetic Resonance (¹H-NMR) Spectroscopy

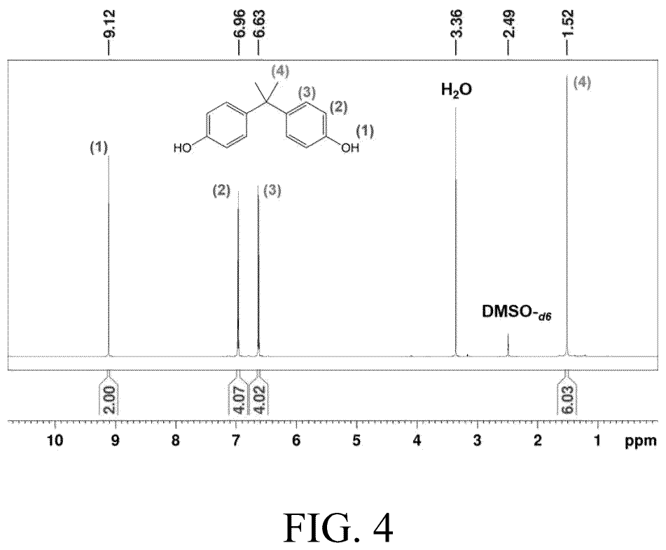

The BPA of EX1 was subjected to ¹H-NMR spectroscopy using an NMR spectrometer (Manufacturer: Bruker Corporation, Model no.: AVIII-500), so as to obtain a ¹H-NMR spectrum as shown in FIG. 4.

Referring to FIG. 4, the ¹H-NMR spectrum showed that the BPA of EX1 had the characteristic peaks of BPA well known to those skilled in the art.

Summarizing the above test results, it is clear that by virtue of the method for manufacturing the BPA of the present disclosure, a high yield of BPA (e.g., 97.6% in the method of EX4) can be obtained. To be specific, by virtue of using the sodium aluminate, which is an inexpensive, easily separable and recyclable catalyst for the heterogeneous catalysis reaction, the depolymerization reaction can be conducted at room temperature under atmospheric pressure, thereby not only reducing production costs and operational complexity but also increasing safety.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for manufacturing a bisphenol A (BPA), comprising:
subjecting a polycarbonate and an aliphatic monohydric alcohol to a depolymerization reaction in the presence of sodium aluminate and a solvent, so as to obtain a first crude product containing the BPA.

2. The method as claimed in claim 1, wherein the BPA is purified from the first crude product by subjecting the first crude product to a solid-liquid separation treatment and a removal treatment in sequence.

3. The method as claimed in claim 1, wherein the solvent is selected from the group consisting of tetrahydrofuran (THF), chloroform, dimethyl carbonate (DMC), dichloromethane (DCM), acetone, acetonitrile, heptane, cyclohexane, and combinations thereof.

4. The method as claimed in claim 1, wherein the aliphatic monohydric alcohol is selected from the group consisting of methanol, ethanol, and a combination thereof.

5. The method as claimed in claim 1, wherein the sodium aluminate is present in an amount ranging from 0.01 parts by weight to 0.20 parts by weight, based on a total amount of the polycarbonate as 1.0 part by weight.

6. The method as claimed in claim 1, wherein the solvent is present in an amount ranging from 0.6 parts by weight to 2.7 parts by weight, based on a total amount of the polycarbonate as 1.0 part by weight.

7. The method as claimed in claim 1, wherein the aliphatic monohydric alcohol is present in an amount ranging from 0.3 parts by weight to 2.4 parts by weight, based on a total amount of the polycarbonate as 1.0 part by weight.

8. The method as claimed in claim 1, wherein the depolymerization reaction is conducted at a temperature ranging from 25.0° C. to 64.7° C.

9. The method as claimed in claim 1, wherein the depolymerization reaction is conducted at a pressure of 1 atm.

10. The method as claimed in claim 1, wherein the depolymerization reaction is conducted for a time period ranging from 0.25 hours to 9.00 hours.
